SEQUENCE LISTING (29 sequences)

SEQ ID NO: 1 - protein, 121 aa, synthetic construct [sequence omitted]

SEQ ID NO: 2 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 3 - protein, 12 aa, synthetic construct
ESKYGPPCPPCP

SEQ ID NO: 4 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 5 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 112 aa, synthetic construct
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKR

SEQ ID NO: 7 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 120 aa, synthetic construct
EVQLVESGGGLVQPGGSLRLSCAASGFTFSRYGMSWVRQAPGKGLELVATINSNGGRTYYPDSVKGRFTISRDNAKNSLYLQMNSLRAEDTAVYYCAREGITTAYAMDYWGQGTTVTVSS

SEQ ID NO: 9 - protein, 98 aa, synthetic construct; identical to SEQ ID NO: 2

SEQ ID NO: 10 - protein, 12 aa, synthetic construct
ESKYGPPCPPCP

SEQ ID NO: 11 - protein, 110 aa, synthetic construct; identical to SEQ ID NO: 4

SEQ ID NO: 12 - protein, 107 aa, synthetic construct; identical to SEQ ID NO: 5

SEQ ID NO: 13 - protein, 112 aa, synthetic construct
DIVMTQSPDSLAVSLGERATINCKASQSVDYDGDSYMHWYQQKPGQPPKLLIYAASILESGVPDRFSGSGSGTDFTLTISSLQAEDVAVYYCQQSNEDPRTFGGGTKVEIKR

SEQ ID NO: 14 - protein, 106 aa, synthetic construct; identical to SEQ ID NO: 7

SEQ ID NO: 15 - protein, 122 aa, synthetic construct
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSS

SEQ ID NO: 16 - protein, 98 aa, synthetic construct; identical to SEQ ID NO: 2

SEQ ID NO: 17 - protein, 12 aa, synthetic construct
ESKYGPPCPPCP

SEQ ID NO: 18 - protein, 110 aa, synthetic construct; identical to SEQ ID NO: 4

SEQ ID NO: 19 - protein, 107 aa, synthetic construct; identical to SEQ ID NO: 5

SEQ ID NO: 20 - protein, 111 aa, synthetic construct
EIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIK

SEQ ID NO: 21 - protein, 106 aa, synthetic construct; identical to SEQ ID NO: 7

SEQ ID NO: 22 - protein, 107 aa, synthetic construct
GQPREPQVYTLPPSREEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLSLGK

SEQ ID NO: 24 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQRGNVFSCSVMHEALHNHYTQKSLSLSLGK

SEQ ID NO: 25 - protein, 107 aa, synthetic construct
GQPREPQVYTLPPSREEMTKNQVSLTCLVKGFYPSDIAVEWESNGQPENNYKTTPPVLDSDGSFFLYSRLTVDKSRWQEGNVFSCSVMHEALHNHYTQKSLSKSLGK

SEQ ID NO: 26 - protein, 107 aa, synthetic construct
GQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLGK

SEQ ID NO: 27 - protein, 107 aa, synthetic construct
GQPREPQVYTLPPSREEMTKNQVSLTCLVKGFYPSDIAVEWQSNGQPENNYKTTPPVLDSDGSFFLYSRLTVDKSRWQEGNVFSCSVMHEALHNHYTQKSLSLSLGK

SEQ ID NO: 28 - protein, 107 aa, synthetic construct
GQPREPQVYTLPPSREEMTKNQVSLTCLVKGFYPSDIAVEWESNGQPENKYKTTPPVLDSDGSFFLYSRLTVDKSRWQEGNVFSCSVMHEALHNHYTQKSLSLSLGK

SEQ ID NO: 29 - protein, 107 aa, synthetic construct
GQPREPQVYTLPPSREEMTKNQVSLTCLVKGFYPSDIAVEWESNGQPENNYKTTPPVLDSDGSFFLYSRLTVDKSRWQRGNVFSCSVMHEALHNHYTQKSLSLSLGK

(19) United States
(12) Patent Application Publication
(10) Pub. No.: US 2026/0217865 A1
(43) Pub. Date: Jul. 30, 2026

(54) IGG FC VARIANTS WITH REDUCED SELF-ASSOCIATION

(71) Applicant: The Regents of the University of Michigan, Ann Arbor, MI (US)

(72) Inventors: Matthew D. Smith, Ann Arbor, MI (US); Peter M. Tessier, Ann Arbor, MI (US); Na-Young Kwon, Ann Arbor, MI (US); Emily Makowski, Ann Arbor, MI (US)

(21) Appl. No.: 19/149,175

(22) PCT Filed: Jan. 18, 2024
(86) PCT No.: PCT/US2024/012003

§ 371 Date: Jul. 18, 2025

Related U.S. Application Data
(60) Provisional application No. 63/480,426, filed on Jan. 18, 2023.

(51) Int. Cl. C07K 16/42; C07K 16/28

(52) U.S. Cl. CPC C07K 16/4291; C07K 16/2866; C07K 16/2878; C07K 2317/24; C07K 2317/52

(57) ABSTRACT

The present disclosure provides polypeptides comprising variant IgG Fc regions, particularly variants which reduced self-association, and compositions and methods of using the polypeptides.

### CROSS-REFERENCE TO RELATED APPLICATIONS

This application claims the benefit of U.S. Provisional Application No. 63/480,426, filed Jan. 18, 2023, the content of which is herein incorporated by reference in its entirety.

### FIELD

The present disclosure relates to polypeptides comprising variant IgG Fc regions, particularly variants which reduced self-association, and compositions and methods of using the polypeptides.

### SEQUENCE LISTING STATEMENT

The content of the electronic sequence listing titled UM_41474_601_SequenceListing.xml (Size: 28,774 bytes; and Date of Creation: Jan. 17, 2024) is herein incorporated by reference in its entirety.

### BACKGROUND

The Fc region of human IgG antibodies provides beneficial attributes for their use in therapeutics, such as a long circulatory half-life and good biochemical and biophysical stability. Fc regions can interact with multiple Fcγ receptor (FcγR) and complement proteins and mediate immune effector functions, important for many therapeutic applications. Most therapeutic antibodies are either IgG1 or IgG4 isotype. IgG4 isotype antibodies, however, show higher self-association relative to IgG1 antibodies with the same variable regions, increasing formulation challenges, particularly at high concentrations due to increases in viscosity, opalescence, aggregation, turbidity, and/or aggregate formation.

### SUMMARY

Disclosed herein are polypeptides comprising a variant IgG Fc region, wherein the variant IgG Fc region comprises one or more amino acid substitutions at positions selected from 355, 362, 382, 390, 419, and 443, in reference to a wild-type or parent IgG Fc region sequence and numbered according to the EU numbering system. In some embodiments, the variant IgG Fc region comprises two or more amino acid substitutions at positions selected from 355, 362, 382, 390, 419, and 443, in reference to a wild-type or parent IgG Fc and numbered according to the EU numbering system.

In some embodiments, the variant IgG Fc region comprises a substitution at position 355. In some embodiments, the variant IgG Fc region comprises an amino acid substitution at position 355 and an amino acid substitution at one of positions 362, 382, 390, 419, or 443. In some embodiments, the amino acid at position 355 of the variant IgG Fc region is substituted with a positively charged amino acid. In some embodiments, the amino acid at position 355 of the variant IgG Fc region is substituted with an arginine.

In some embodiments, the variant IgG Fc region comprises an amino acid substitution at position 419. In some embodiments, the variant IgG Fc region comprises an amino acid substitution at position 419 and an amino acid substitution at one of positions 355, 362, 382, 390, or 443. In some embodiments, the amino acid at position 419 of the variant IgG Fc region is substituted with a non-charged or positively charged amino acid. In some embodiments, the amino acid at position 419 of the variant IgG Fc region is substituted with an arginine or glutamine.

In some embodiments, the amino acid at position 362 of the variant IgG Fc region is substituted with a positively charged amino acid. In some embodiments, the amino acid at position 362 of the variant IgG Fc region is substituted with a lysine.

In some embodiments, the amino acid at position 382 of the variant IgG Fc region is substituted with a glutamine. In some embodiments, the amino acid at position 390 of the variant IgG Fc region is substituted with a positively charged amino acid. In some embodiments, the amino acid at position 390 of the variant IgG Fc region is substituted with a lysine. In some embodiments, the amino acid at position 443 of the variant IgG Fc region is substituted with a positively charged amino acid. In some embodiments, the amino acid at position 443 of the variant IgG Fc region is substituted with an arginine or lysine.

In some embodiments, the variant IgG Fc region comprises amino acid substitutions selected from: 355R; 419Q; 419R; 355R and 443K or 443R; 355R and 419Q or 419R; 355R and 362K; 355R and 382Q; 355R and 390K; 419Q and 443K or 443R; 419Q and 362K; 419Q and 382Q; and 419Q and 390K.

In some embodiments, the variant IgG Fc region is derived from a human IgG Fc region. In some embodiments, the variant IgG Fc region is an IgG4 Fc region.

In some embodiments, the polypeptide further comprises a target molecule binding domain. In some embodiments, the polypeptide is an antibody or an Fc fusion protein. In some embodiments, the antibody is a monoclonal antibody, a humanized antibody, a chimeric antibody, a recombinant antibody, a monospecific antibody, a bispecific antibody, or a multi-specific antibody.

In some embodiments, the polypeptide is conjugated to a drug, a toxin, a detectable label, a tag, or a combination thereof.

In some embodiments, the polypeptide has reduced self-association characteristics.

Further disclosed herein are compositions comprising the disclosed polypeptides and a pharmaceutically acceptable carrier. In some embodiments, the composition is a liquid composition. In some embodiments, the composition has a pH of about 5 to about 7.

In some embodiments, the compositions comprise at least 10 mg/mL of a disclosed polypeptide.

In some embodiments, the compositions comprise a buffering agent. In some embodiments, the buffering agent is an amino acid. In some embodiments, the amino acid comprises histidine.

In some embodiments, the compositions further comprise a tonicity agent, an antioxidant, a stabilizer, or a combination thereof. In some embodiments, the compositions further comprise an adjuvant.

Additionally disclosed are methods of decreasing the self-association of a polypeptide comprising an IgG Fc region comprising introducing one or more amino acid substitutions in the IgG Fc region at positions selected from 355, 362, 382, 390, 419, 443, in reference to a wild-type IgG Fc and numbered according to the EU numbering system.

In some embodiments, the one or more amino acid substitutions comprise a substitution at position 355. In some embodiments, the one or more amino acid substitutions comprise an amino acid substitution at position 355 and an amino acid substitution at one of positions 362, 382, 390, 419, or 443. In some embodiments, the amino acid at position 355 of the IgG Fc region is substituted with a positively charged amino acid. In some embodiments, the amino acid at position 355 of the IgG Fc region is substituted with an arginine.

In some embodiments, the one or more amino acid substitutions comprise an amino acid substitution at position 419. In some embodiments, the one or more amino acid substitutions comprise an amino acid substitution at position 419 and an amino acid substitution at one of positions 355, 362, 382, 390, or 443. In some embodiments, the amino acid at position 419 of the IgG Fc region is substituted with a non-charged or positively charged amino acid. In some embodiments, the amino acid at position 419 of the IgG Fc region is substituted with an arginine or glutamine.

In some embodiments, the amino acid at position 362 of the IgG Fc region is substituted with a positively charged amino acid. In some embodiments, the amino acid at position 362 of the IgG Fc region is substituted with a lysine.

In some embodiments, the amino acid at position 382 of the IgG Fc region is substituted with a glutamine. In some embodiments, the amino acid at position 390 of the IgG Fc region is substituted with a positively charged amino acid. In some embodiments, the amino acid at position 390 of the IgG Fc region is substituted with a lysine. In some embodiments, the amino acid at position 443 of the IgG Fc region is substituted with a positively charged amino acid. In some embodiments, the amino acid at position 443 of the IgG Fc region is substituted with an arginine or lysine.

In some embodiments, the IgG Fc region comprises amino acid substitutions selected from: 355R; 419Q; 419R; 355R and 443K or 443R; 355R and 419Q or 419R; 355R and 362K; 355R and 382Q; 355R and 390K; 419Q and 443K or 443R; 419Q and 362K; 419Q and 382Q; and 419Q and 390K.

In some embodiments, the IgG Fc region is derived from a human IgG Fc region. In some embodiments, the IgG Fc region is an IgG4 Fc region.

Also disclosed are methods of treating a disease or disorder in a subject, comprising administering to the subject an effective amount of a polypeptide, composition, or nucleic acid, as described herein.

Other aspects and embodiments of the disclosure will be apparent in light of the following detailed description and accompanying figures.

### BRIEF DESCRIPTION OF THE DRAWINGS

FIG. 1 is a graph of charge-stabilized self-interaction nanoparticle spectroscopy (CS-SINS) measurements of omalizumab and omalizumab with the C_{H}3 domain swapped with that of wild-type or mutant IgG4. Omalizumab WT IgG1 and IgG4, n=9; Mut 1-3, n=6; and Mut4-6, 8-15 (n=3).

FIGS. 2A and 2B are CS-SINS results for omalizumab IgG4 variants at pH 6 (10 mM histidine) (FIG. 2A) and pH 5 (10 mM acetate) (FIG. 2B). For FIG. 2A, N=3-9 and error bars are standard deviations. For FIG. 2B, N=4-9 and error bars are standard deviations.

FIGS. 3A-3C show the biophysical analysis of select omalizumab IgG4 variants. FIG. 3A shows the normalized expression level. FIG. 3B shows the % monomer after 1-step Protein A purification. FIG. 3C shows the melting temperature (first unfolding transition). The number of data replicates for FIG. 3A is 2-3, FIG. 3B is 2-3 and FIG. 3C is 2, and the error bars are standard deviations

FIG. 4 is the analytical size-exclusion chromatography analysis of select omalizumab IgG4 variants after two-step purification. The MW markers are in kDa units.

FIG. 5 is a graph showing that the Fc mutations increase the solubility of omalizumab IgG4. The IgG solubility was evaluated for WT omalizumab and Fc variants using an ultracentrifugation assay. Antibodies (~1 mg per IgG) were concentrated using 10 kDa MWCO filters from 1 mL to ~0.03-0.05 mL, and the resulting soluble concentrations were evaluated using UV absorbance measurements. The experiments were performed at pH 6 (10 mM histidine) at 4° C.

FIGS. 6A and 6B are CS-SINS results for revdofilimab IgG4 variants at pH 6 (10 mM histidine) (FIG. 6A) and pH 5 (10 mM acetate) (FIG. 6B). For FIG. 6A, N=3-8 and error bars are standard deviations. For FIG. 6B, N=3-6 and error bars are standard deviations.

FIGS. 7A-7C show the biophysical analysis of select revdofilimab IgG4 variants. FIG. 7A shows the normalized expression level. FIG. 7B shows the % monomer after 1-step Protein A purification. FIG. 7C shows the melting temperature (first unfolding transition). The number of data replicates for FIG. 7A is 2-3, FIG. 7B is 2-3 and FIG. 7C is 2, and the error bars are standard deviations

FIG. 8 is the analytical size-exclusion chromatography analysis of select revdofilimab IgG4 variants after two-step purification. The MW markers are in kDa units.

FIGS. 9A and 9B are CS-SINS results for cabiralizumab IgG4 variants at pH 6 (10 mM histidine) (FIG. 9A) and pH 5 (10 mM acetate) (FIG. 9B). For FIG. 9A, N=3-6 and error bars are standard deviations. For FIG. 9B, N=3-5 and error bars are standard deviations.

FIGS. 10A-10C show the biophysical analysis of select cabiralizumab IgG4 variants. FIG. 10A shows the normalized expression level. FIG. 10B shows the % monomer after 1-step Protein A purification. FIG. 10C shows the melting temperature (first unfolding transition). The number of data replicates for FIG. 10A is 2-4, FIG. 10B is 3-5 and FIG. 10C is 2, and the error bars are standard deviations

FIG. 11 is the analytical size-exclusion chromatography analysis of select cabiralizumab IgG4 variants after two-step purification. The MW markers are in kDa units.

### DETAILED DESCRIPTION

Disclosed herein are variant IgG Fc regions which reduce self-association and/or increase solubility. Herein, the self-association of a known antibody, as measured by CS-SINS, was reduced when variant C_{H}3 domains from IgG4 were swapped with the preferred IgG1 isotype C_{H}3 domain, whereas the solubility was increased.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids include, for example, pyrolysine (found in methanogenic organisms and other eukaryotes), selenocysteine (present in many non-eukaryotes as well as most eukaryotes), and N-formylmethionine (encoded by the start codon AUG in bacteria, mitochondria, and chloroplasts). “Unnatural” or “non-natural” amino acids are non-proteinogenic amino acids (e.g., those not naturally encoded or found in the genetic code) that either occur naturally or are chemically synthesized. Over 140 unnatural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid. According to certain embodiments, a peptide inhibitor comprises an intramolecular bond between two amino acid residues present in the peptide inhibitor. It is understood that the amino acid residues that form the bond will be altered somewhat when bonded to each other as compared to when not bonded to each other. Reference to a particular amino acid is meant to encompass that amino acid in both its unbonded and bonded state. For example, the amino acid residue homoSerine (hSer) or homoSerine (Cl) in its unbonded form may take the form of 2-aminobutyric acid (Abu) when participating in an intramolecular bond according to the present invention.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14 (2), (1975). To the extent that the names and abbreviations of amino acids and aminoacyl residues employed in this specification and appended claims differ from those suggestions, they will be made clear to the reader.

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D)Phe, D-Phe, or ^{D}F for the D isomeric form of Phenylalanine). Amino acid residues in the D isomeric form can be substituted for any L-amino acid residue, as long as the desired function is.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and 8Ado (8-amino-3,6-dioxaoctanoic acid), Abu (2-amino butyric acid), βhPro (β-homoproline), βhPhe (β-homophenylalanine) and Bip (β,β diphenylalanine), and Ida (Iminodiacetic acid).

“Antibody” and “antibodies” as used herein refer to monoclonal antibodies, polyclonal antibodies, monospecific antibodies (e.g., which can either be monoclonal, or may also be produced by other means than producing them from a common germ cell), multi-specific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′) 2 fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25 (11): 1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass.

Typically, an immunoglobulin or antibody is a protein that comprises at least one complementarity determining region (CDR). The CDRs form the “hypervariable region” of an antibody, which is responsible for antigen binding. “CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1,” “CDR2,” and “CDR3,” for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “Fc region,” as used herein, generally refers to a dimer complex comprising the C-terminal polypeptide sequences of an immunoglobulin heavy chain, wherein a C-terminal polypeptide sequence is that which is obtainable by papain digestion of an intact antibody. Although the boundaries of the Fc sequence of an immunoglobulin heavy chain might vary, the human IgG heavy chain Fc sequence is from about position 226 or 230 to the carboxyl terminus of the Fc sequence. Unless otherwise specified herein, numbering of amino acid residues in the Fc region or constant region is according to the EU numbering system, also called the EU index. The Fc sequence of an immunoglobulin generally comprises two constant domains, a C_{H}2 domain and a C_{H}3 domain, and optionally comprises a C_{H}4 domain. An Fc region may be obtained from any suitable immunoglobulin, such as human IgG1, IgG2, IgG3, or IgG4 subtypes, IgA, IgE, IgD or IgM.

The “C_{H}2 domain” or “C_{H}2 domain” of a human IgG Fc region usually extends from about amino acid 231 to about amino acid 340. The C_{H}2 domain is unique in that it is not closely paired with another domain. Rather, two N-linked branched carbohydrate chains are interposed between the two C_{H}2 domains of an intact native IgG molecule. It has been speculated that the carbohydrate may provide a substitute for the domain-domain pairing and help stabilize the C_{H}2 domain (Burton, Molec. Immunol. 22 (1985) 161-206). The “C_{H}3 domain” or “C_{H}3 domain” comprises the stretch of residues C-terminal to a C_{H}2 domain in an Fc region (e.g., from about position 341 to about position 447).

A “wild-type Fc region sequence” comprises an amino acid sequence identical to the amino acid sequence of an Fc region found in nature.

A “parent Fc region sequence” as used herein refers to an Fc region that is unmodified in relationship to the position being modified in the variant and is subsequently modified to generate a variant. For example, some parent Fc region sequences have the amino acids as in the wild-type Fc region sequence in those positions of interest in the variant Fc region sequences (e.g., 355, 362, 382, 390, 419, and 443, according to the EU numbering system). The parent Fc region sequence may be that of the wild-type Fc region, or a variant or engineered version of the wild-type Fc region.

A “variant Fc region” comprises an amino acid sequence which differs from that of a wild-type or parent Fc region sequence by virtue of at least one amino acid substitution(s).

As used herein, an “Fc fusion protein” refers to a protein wherein one or more polypeptides are operably linked to an Fc region The term “Fc fusion protein” is synonymous with the terms “immunoadhesin,” “Ig fusion,” “Ig chimera,” and “receptor globulin.” An Fc fusion protein comprises an Fc region of an immunoglobulin (e.g., Fc domain of IgG4) that is operably linked to a polypeptide, such as a receptor, an adhesion molecule, a ligand, an enzyme, a cytokine, or some other protein domain, wherein the polypeptide is capable of binding to a particular target (e.g., a ligand, a receptor, a substrate etc.). Examples of Fc fusion proteins include but are not limited to the Fc fusion proteins described in U.S. Pat. Nos. 5,843,725; 6,018,026; 6,291,212; 6,291,646; 6,300,099; 6,323,323; PCT WO 00/24782; and in (Chamow et al., 1996, Trends Biotechnol 14:52-60; Ashkenazi et al., 1997, Curr Opin Immunol 9:195-200).

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5:511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352:624-628 (1991)); and Marks et al., J. Mol. Biol., 222:581-597 (1991)), or produced from transgenic mice carrying a fully human immunoglobulin system (see, e.g., Lonberg, Nat. Biotechnol., 23 (9): 1117-25 (2005), and Lonberg, Handb. Exp. Pharmacol., 181:69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

The term “monospecific” antibody as used herein denotes an antibody that has one or more binding sites each of which bind to the same epitope of the same antigen.

The term “bispecific” antibody as used herein denotes an antibody that has at least two binding sites each of which bind to different epitopes of the same antigen or a different antigen.

The term “multi-specific” antibody as used herein denotes an antibody that has binding specificities for at least two different sites.

A “polypeptide,” “protein,” or “peptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide”, “protein,” and “peptide” are used interchangeably herein.

As used herein, “position” refers to a location in the sequence of a protein. Positions may be numbered sequentially, or according to an established format.

As used herein, “EU index” or “EU numbering” refers to the numbering of the EU antibody (Edelman, et al., Proc Natl Acad Sci USA 63 (1969) 78-85, hereby entirely incorporated by reference). “Kabat index” or “Kabat numbering” refers to the numbering according to Kabat et al., Sequences of Proteins of Immunological Interest, 5th Ed. Public Health Service, National Institutes of Health, Bethesda, Md. (1991), hereby entirely incorporated by reference).

As used herein, the terms “providing,” “administering,” and “introducing” are used interchangeably herein and refer to the placement of the polypeptides and/or compositions of the present disclosure into a subject by a method or route which results in at least partial localization to a desired site.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

A “target molecule” or “target” refers to a moiety recognized by a binding domain of the disclosed polypeptides. For example, if the polypeptide is an antibody, then the target may be epitopes on a single molecule or on different molecules, or a pathogen or a tumor cell, depending on the context. Similarly, if the polypeptide is a receptor-Fc fusion protein the target would be the cognate binding partner for the receptor. One skilled in the art will appreciate that the target is determined by the specificity of the target binding domain and that different binding domains may recognize different targets. A target preferably binds to a polypeptide provided herein with affinity higher than 1 μM K_{d}. Examples of target molecule classes include, but are not limited to, serum soluble proteins and/or their receptors, such as cytokines and/or cytokine receptors, adhesins, growth factors and/or their receptors, hormones, viral particles (e.g., RSV F protein, CMV, Staph A, influenza, hepatitis C virus), microorganisms (e.g., bacterial cell proteins, fungal cells), adhesins, CD proteins and their receptors.

As used herein, “treat,” “treating,” and the like means a slowing, stopping, or reversing of progression of a disease or disorder when provided a polypeptide or composition described herein to an appropriate subject. The term also includes a reversing of the progression of such a disease or disorder to a point of eliminating or greatly reducing the disease. As such, “treating” means an application or administration of the polypeptides or compositions described herein to a subject, where the subject has a disease or a symptom of a disease, where the purpose is to cure, heal, alleviate, relieve, alter, remedy, ameliorate, improve, or affect the disease or symptoms of the disease.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Polypeptides

The present disclosure provides polypeptides comprising a variant IgG Fc region. The variant IgG Fc region may comprise one or more amino acid substitutions. The variant IgG Fc region may confer a variety of different characteristics to the Fc region or the corresponding polypeptide, preferably characteristics which result in improved or superior functionality (e.g., purification ease and yield, formulation ease, administration, effectiveness, solubility, stability).

In some embodiments, the variant IgG Fc region, and therefore, the polypeptide, has reduced self-association characteristics. Reduced self-association characteristics include reduced viscosity, reduced opalescence, decreased phase separation, reduction in aggregates or aggregation. In some embodiments, the variant IgG Fc region facilitates easier purification with higher yields of recombinantly produced forms of the polypeptide.

The variants may comprise one or more amino acid substitutions in comparison to a wild-type or parent IgG Fc region sequence. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

Herein, substitutions or substitution positions separated by a “/” represent a polypeptide having each of the substitution or a substitution at each of the recited positions.

In some embodiments, the variant IgG Fc region comprises one or more amino acid substitutions (e.g., one, two, three, four, five, or six) at positions selected from 355, 362, 382, 390, 419, and 443, in reference to a wild-type or parent IgG Fc region sequence and numbered according to the EU numbering system. In some embodiments, the variant IgG Fc region comprises two or more amino acid substitutions (e.g., two, three, four, five, or six) at positions selected from 355, 362, 382, 390, 419, and 443, in reference to a wild-type or parent IgG Fc region sequence and numbered according to the EU numbering system.

Accordingly, in some embodiments, the variant IgG Fc region comprises one or more amino acid substitutions (e.g., one, two, three, four, five, or six) at positions selected from 376, 385, 407, 418, 450, and 474, in reference to a wild-type or parent IgG Fc region sequence and numbered according to the Kabat numbering system. In some embodiments, the variant IgG Fc region comprises two or more amino acid substitutions (e.g., two, three, four, five, or six) at positions selected from 376, 385, 407, 418, 450, and 474, in reference to a wild-type or parent IgG Fc region sequence and numbered according to the Kabat numbering system. Throughout the disclosure, unless otherwise stated, any reference to particular substitutions or combinations of substitutions are referred to by referencing the EU numbering scheme and those disclosures also encompass those coordinating substitutions or combinations of substitutions represented by the Kabat numbering scheme or another coordinating numbering scheme.

In some embodiments, the variant IgG Fc region comprises a substitution at position 355. In select embodiments, the variant IgG Fc region comprises a substitution at position 355 and at least one (e.g., one, two, three, four, or five) substitution at positions 362, 382, 390, 419, or 443. In some embodiments, the amino acid at position 355 of the variant IgG Fc region is substituted with a positively charged amino acid (e.g., arginine (R), histidine (H), lysine (K)). In select embodiments, the amino acid at position 355 of the variant IgG Fc region is substituted with an arginine.

In some embodiments, the variant IgG Fc region comprises an amino acid substitution at position 419. In select embodiments, the variant IgG Fc region comprises a substitution at position 419 and at least one (e.g., one, two, three, four, or five) substitution at positions 355, 362, 382, 390, or 443. In some embodiments, the amino acid at position 419 of the variant IgG Fc region is substituted with a non-charged (e.g., polar uncharged amino acids, non-polar uncharged amino acids) or positively charged amino acid. In select embodiments, the amino acid at position 419 of the variant IgG Fc region is substituted with an arginine. In select embodiments, the amino acid at position 419 of the variant IgG Fc region is substituted with a glutamine.

In some embodiments, the variant IgG Fc region comprises an amino acid substitution at position 362, alone or with one or more of the other disclosed substitutions. In some embodiments, the amino acid at position 362 of the variant IgG Fc region is substituted with a positively charged amino acid. In select embodiments, the amino acid at position 362 of the variant IgG Fc region is substituted with a lysine.

In some embodiments, the variant IgG Fc region comprises an amino acid substitution at position 382, alone or with one or more of the other disclosed substitutions. In some embodiments, the amino acid at position 382 of the variant IgG Fc region is substituted with a non-charged amino acid. In select embodiments, the amino acid at position 382 of the variant IgG Fc region is substituted with a glutamine.

In some embodiments, the variant IgG Fc region comprises an amino acid substitution at position 390, alone or with one or more of the other disclosed substitutions. In some embodiments, the amino acid at position 390 of the variant IgG Fc region is substituted with a positively charged amino acid. In select embodiments, the amino acid at position 390 of the variant IgG Fc region is substituted with a lysine.

In some embodiments, the variant IgG Fc region comprises an amino acid substitution at position 443, alone or with one or more of the other disclosed substitutions. In some embodiments, the amino acid at position 443 of the variant IgG Fc region is substituted with a positively charged amino acid. In some embodiments, the amino acid at position 443 of the variant IgG Fc region is substituted with an arginine. In some embodiments, the amino acid at position 443 of the variant IgG Fc region is substituted with a lysine.

In some embodiments, the variant IgG Fc region comprises amino acid substitutions selected from: 355R; 419Q; 419R; 355R and 443K or 443R; 355R and 419Q or 419R; 355R and 362K; 355R and 382Q; 355R and 390K; 419Q and 443K or 443R; 419Q and 362K; 419Q and 382Q; and 419Q and 390K.

In addition to the substitutions described herein, the variant IgG Fc region may further contain other amino acid substitutions, insertions, or deletions.

In some embodiments, the variant IgG Fc region is derived from a human IgG Fc region. In some embodiments, the variant IgG Fc region is derived from a human IgG1, IgG2, IgG3, or IgG4 Fc region. In some embodiments, the variant IgG Fc region is derived from an IgG4 Fc region. In select embodiments, the variant IgG Fc region is derived from a human IgG4 Fc region.

In some embodiments, the polypeptide may consist or consist essentially of the variant IgG Fc region. Conversely, in some embodiments, the polypeptide may comprise other protein domains, polypeptides, or moieties. In select embodiments, the polypeptide comprises a target molecule binding domain. The type and nature of the binding domain will determine the specificity for the target molecule, as different binding domains may be engineered to recognize a variety of different target molecules. Examples of target molecule classes include, but are not limited to, serum soluble proteins and/or their receptors, such as cytokines and/or cytokine receptors, adhesins, growth factors and/or their receptors, hormones, viral particles, microorganisms or microorganismal proteins and nucleic acids, and adhesins.

In some embodiments, the polypeptide is an antibody. In some embodiments, the antibody is a monoclonal antibody, a humanized antibody, a chimeric antibody, a recombinant antibody, a monospecific antibody, a bispecific antibody, or a multi-specific antibody.

In some embodiments, the polypeptide is an Fc fusion protein. Fc fusion proteins are composed of an immunoglobin Fc domain that is directly linked to another peptide. The fused partner can be any other proteinaceous molecule of interest, such as a ligand that activates upon interaction with a cell-surface receptor, a peptidic antigen (Ag) against a challenging pathogen, a signaling molecule (e.g., cytokine), extracellular domain of a pattern recognition receptor (PRR), or a bait protein to identify binding partners (e.g., as assembled in a protein microarray). Most frequently though, the fused partners have significant therapeutic potential, and are attached to an Fc-domain to endow the hybrids with a number of additional beneficial biological and pharmacological properties (e.g., improved solubility, stability, pharmacokinetics).

The polypeptides disclosed herein can be linked or conjugated to molecules useful as detectable labels (fluorophores, chromophores, and the like), tags (e.g., 3×FLAG tag, an HA tag, a Myc tag, and the like), drugs (e.g., cytotoxic drugs), toxins or toxic proteins (e.g., diphtheria toxin, Pseudomonas exotoxin A), or the like. A polypeptide as disclosed herein may be linked or conjugated to another protein or protein domain that provides for tagging or visualization (e.g., GFP) or for entry into a cell (e.g., protein transduction domains or PTDs, also known as a CPP, a cell penetrating peptide) or cellular compartment (e.g., the nucleus with a nuclear localization sequence as described elsewhere herein).

The variant IgG Fc regions of the present invention can be constructed by mutating the DNA sequences that encode the corresponding parent IgG Fc region (e.g., a wild-type variant IgG Fc region), such as by using techniques commonly referred to as site-directed mutagenesis. Alternatively, polypeptides having Fc regions (e.g., antibodies or Fc fusion proteins) can be modified to comprise IgG Fc regions of the present invention by mutating the IgG Fc region, as described above for variant IgG Fc regions, or by swapping the DNA sequences that encode the corresponding parent IgG Fc region (e.g., a wild-type variant IgG Fc region) with sequences that encode a variant IgG Fc region as described herein, such as by using common molecular cloning technique.

For example, antibodies which could benefit from such modifications may include, but are not limited to: natalizumab, gemtuzumab ozogamicin, nivolumab, pembrolizumab, rozanolixizumab, talquetamab, lebrikizumab, mirikizumab, tislelizumab, sintilimab, toripalimab, retifanlimab, narsoplimab, teclistamab, relatlimab, sutimlimab, tralokinumab, evinacumab, dostarlimab, ravulizumab, cemiplimab, galcanezumab, ibalizumab, emicizumab, inotuzumab ozogamicin, dupilumab, reslizumab, ixekizumab, eculizumab, zimberelimab, sugemalimab, narlumosbart, serplulimab, rozanolixizumab, pucotenlimab, olokizumab, mirikizumab, lebrikizumab, geptanolimab, concizumab, concizumab, adebrelimab, tralokinumab, camrelizumab, sabatolimab, magrolimab, spartalizumab, and natalizumab.

Additional antibodies which could benefit from such modifications may include: rituximab, trastuzumab, alemtuzumab, bevacizumab, cetuximab, ofatumumab, ipilimumab, pertuzumab, obinutuzumab, ramucirumab, dratumumab, elotuzumab, dinutuximab, necitumumab, loaratumab, atezolizumab, panitumumab, durvalumab, tiragolumab, ustekinumab, vibostolimab, tremelimumab, isatuximab, pamrevlumab, epcoritamab, trastuzumab duocarmazine, donanemab, lecanemab, penpulimab, omburtamab, inolimomab, teplizumab, ublituximab, mirvetuximab soravtansine, nirsevimab, spesolimab, mosunetuzumab, tixagevimab cilgavimab, faricimab, sotrovimab, regdanvimab, casirivimab, imdevimab, tezepelumab, tisotumab vedotin, amivantamab, anifromlumab, loncastuximab tesirine, bimekizumab, aducanumab, ansuvimab, margetuximab, naxitamab, atoltivimab, maftivimab, odesivimabebgn, belantamab mafodotin, tafasitamab, satralizumab, inebilizumab, sacituzumab govitecan, teprotumumab, istuximab, eptinezumab, trastuzumab deruxtecan, enfortumab vedotin, crizanlizumab, polatuzumab vedotin, risankizumab, romosozumab, ravulizumab, emapalumab, fremanezumab, moxetumamomab, lanadelumab, mogamulizumab, erenumab, tildrakizumab, burosumab, curalumab, benralizumab, ocrelizumab, guselkumab, sarilumab, avelumab, brodalumab, bezlotoxumab, olaratumab, obiltoxaximab, daratumumab, elltuzumab, alirocumab, mepolizumab, evolocumab, secukinumab, vedolizumab, cabiralizumab, revdofilimab, siltuximab, ado-trastuzumab emtansine, raxibacumab, brentuximab vedotin, velimumab, denosumab, tocilizumab, canakinumab, golimumab, ranibizumab, beacizumab, efalizumab, omalizumab, ibritumomab tiuxetan, adalimumab, infliximab, palivizumab, basilizimab, daclizumab, abciximab, belantamab mafodotin, disitamab, and edrecolomab.

As such, in various other embodiments, the present invention provides a recombinant nucleic acid encoding a DNA polymerase as described herein, a vector comprising the recombinant nucleic acid, and a host cell transformed with the vector.

Accordingly, the present disclosure further provides methods for decreasing the self-association of a polypeptide comprising an IgG Fc region. The methods comprise introducing any of the above disclosed substitutions in a parent (e.g., wild-type) IgG Fc region sequence. In some embodiments, the polypeptide is an antibody. In some embodiments, the antibody is a monoclonal antibody, a humanized antibody, a chimeric antibody, a recombinant antibody, a monospecific antibody, a bispecific antibody, or a multi-specific antibody. In some embodiments, the polypeptide is an Fc fusion protein. In some embodiments, the IgG Fc region is derived from a human IgG Fc region. In some embodiments, the IgG Fc region is an IgG4 Fc region.

The nucleic acids or vectors may be used to propagate the polypeptides disclosed herein in an appropriate cell and/or to allow expression from the segment (e.g., an expression vector). The person of ordinary skill in the art would be aware of the various vectors available for propagation and expression of a nucleic acid sequence. The vector(s) and nucleic acid(s) can be introduced into a cell that is capable of expressing the polypeptide encoded thereby, including any suitable prokaryotic or eukaryotic cell.

In one embodiment, a DNA segment encoding the polypeptides disclosed herein is contained in a plasmid vector that allows expression of the protein and subsequent isolation and purification of the protein produced by the recombinant vector. Accordingly, the polypeptides disclosed herein can be purified following expression, obtained by chemical synthesis, or obtained by recombinant methods.

To construct cells that express the polypeptides disclosed herein, expression vectors for stable or transient expression of the polypeptide may be constructed via methods as described herein or known in the art and introduced into cells. For example, nucleic acids encoding the polypeptide may be cloned into a suitable expression vector, such as a plasmid or a viral vector in operable linkage to a suitable promoter.

Vectors according to the present disclosure can be transformed, transfected, or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art.

### 3. Compositions

Further disclosed herein are compositions comprising the polypeptides described herein. The compositions may comprise excipients or pharmaceutically acceptable carriers. The choice of excipients or pharmaceutically acceptable carriers will depend on factors including, but not limited to, the particular mode of administration, the effect of the excipient on solubility and stability, and the nature of the dosage form. The compositions of the present invention will be readily apparent to those skilled in the art. Techniques and formulations may be found, for example, in Remington's Pharmaceutical Sciences, 19th Edition (Mack Publishing Company, 1995).

The term “pharmaceutically acceptable carrier,” as used herein, means a non-toxic, inert solid, semi-solid or liquid filler, diluent, encapsulating material, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as releasing agents, coating agents, preservatives and antioxidants can also be present in the composition, according to the judgment of the formulator.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis).

In some embodiments, the composition is a liquid or aqueous composition, particularly useful for parenteral administration (e.g., intravenous, subcutaneous, intramuscular).

In some embodiments, the composition comprises at least 10 mg/mL of the polypeptide. The composition may comprise at 10-500 mg/mL (e.g., 10-400, 20-400, 50-300, or 100-300 mg/mL) of the polypeptide. The composition may comprise at least 10 mg/mL, at least 20 mg/mL, at least 30 mg/mL, at least 40 mg/mL, at least 50 mg/mL, at least 60 mg/mL, at least 70 mg/mL, at least 80 mg/mL, at least 90 mg/mL, at least 100 mg/mL, at least 125 mg/mL, at least 150 mg/mL, at least 175 mg/mL, at least 200 mg/mL, at least 250 mg/mL, at least 300 mg/mL or more of the polypeptide. In select embodiments, the composition comprises at least 100 mg/mL of the polypeptide.

In some embodiments, the composition comprises a buffering agent. The buffer serves to maintain a physiologically suitable pH. In addition, the buffer can serve to enhance isotonicity and chemical stability of the composition. Generally, the composition has a physiologically suitable pH. In some embodiments, the composition has a pH of about 5 to about 7, about 5.5 to about 6.5, preferably about 6.0 to about 6.5. In select embodiments, the composition has a pH of about 6. Ranges intermediate to the above recited pH levels, for example, about pH 5.2 to about pH 6.3, preferably 6.0 or pH 6.2, are also encompassed. The pH may be adjusted as necessary. For example, HCl may be added as necessary to adjust the pH to desired levels.

Exemplary buffering agents include, but are not limited, to succinate (sodium or phosphate), amino acids (e.g., histidine, arginine, methionine), phosphate (sodium or potassium), Tris(tris(hydroxymethyl)aminomethane), diethanolamine, citrate, other organic acids, and combinations thereof. In some embodiments, the compositions comprise an amino acid in an amount sufficient to maintain the compositions at a physiologically suitable pH.

In some embodiments, the amino acid is histidine. Histidine has buffering capabilities in the physiological pH range due to the imidazole group. Examples of histidine buffer agents include but are not limited to histidine chloride, histidine acetate, histidine phosphate, histidine sulfate, histidine succinate, etc. In select embodiments, the buffer is L-histidine (base). In select embodiments, the buffer is L-histidine monochloride monohydrate. In select embodiments, the buffer is a mixture of L-histidine (base) and L-histidine monochloride monohydrate. Histidine-based buffers can further contain other amino acids (e.g., arginine, methionine, and the like).

The buffering agent may be present in a concentration from about 0.1 mM to about 50 mM, from about 0.1 mM to about 40 mM, from about 0.1 mM to about 30 mM, about 0.1 mM to about 25 mM, from about 0.1 mM to about 20 mM, or from about 5 mM to about 15 mM, preferably 5 mM or 10 mM. In select embodiments, the buffering agent may be present at about 6 mM, about 7 mM, about 8 mM, about 9 mM, about 10 mM, about 11 mM, about 12 mM, about 13 mM, about 14 mM, or about 15 mM. In an exemplary embodiments, the buffer agent is present at about 10 mM.

In select embodiments, the composition comprises histidine as the buffering agent at concentrations from about 0.1 mM to about 25 mM (e.g., about 1 mM, about 5 mM, about 10 mM, about 15 mM, and the like).

In some embodiments, the composition further comprises a tonicity agent, an antioxidant, a stabilizer, or a combination thereof.

A tonicity agent contributes to maintaining the isotonicity of the composition and preserving the level, ratio, or proportion of the polypeptide. Isotonic solutions possess the same osmotic pressure as blood plasma, and so can be intravenously infused into a subject without changing the osmotic pressure of the subject's blood plasma. The tonicity agent is present in an amount sufficient to render the composition suitable for intravenous administration. Exemplary tonicity agents include, but are not limited to, CaCl₂), NaCl, MgCl₂, lactose, sorbitol, sucrose, mannitol, trehalose, raffinose, polyethylene glycol, hydroxyethyl starch, glycine, and mixtures thereof. The tonicity agent may be present at about 2% to about 6% w/v (e.g., about 3% to about 5% w/v) or at about 20 mg/ml to about 60 mg/ml (e.g., about 30 mg/ml to about 50 mg/ml, about 35 mg/ml to about 45 mg/ml).

An antioxidant preserves the composition by preventing oxidation. Exemplary antioxidants include, but are not limited to, phenolic antioxidants (for example, eugenol, carnosic acid, caffeic acid, BHT (butylated hydroxyanisol), gallic acid, tocopherols, tocotrienols and flavonoid antioxidants (such as myricetin and fisetin)), polyenes (for example, retinoic acid), unsaturated sterols (for example, Δ5-avenosterol), organosulfur compounds (for example, allicin), terpenes (for example, geraniol, abietic acid) and amino acid antioxidants (for example, methionine, cysteine, carnosine). In some embodiments, the antioxidant may further comprise GLA (gamma-linolenic acid)-lipoic acid, DHA (docosahexaenoic acid)-lipoic acid, GLA-tocopherol, di-GLA-3,3′-thiodipropionic acid and in general any of, for example, GLA, DGLA (dihomo-gamma-linolenic acid), AA (arachidonic acid), SA (salicylic acid), EPA (eicosapentaenoic acid) or DHA (docosahexaenoic acid) with any natural or synthetic antioxidant, as described above, with which they can be chemically linked. The antioxidant may be present in the composition from about 0.1 mM to about 50 mM (e.g., about 0.1 mM to about 40 mM, about 0.1 mM to about 30 mM, about 0.1 mM to about 20 mM, or about 5 mM to about 15 mM).

A stabilizer interacts and stabilizes biological molecules and/or general pharmaceutical excipients in a composition. In certain embodiments, stabilizers may be used in conjunction with lower temperature storage. Stabilizers generally protect the polypeptide from air/solution interface induced stresses and solution/surface induced stresses, which may otherwise result in protein aggregation. The stabilizer may include, but is not limited to, glycerin, polysorbates such as polysorbate 80, dicarboxylic acids, oxalic acid, succinic acid, adipic acid, fumaric acid, phthalic acids, and combinations thereof. The stabilizer may be present in the composition from about 0.001% w/v to about 0.01% w/v (e.g., about 0.001% w/v to about 0.009% w/v, or about 0.003% w/v to about 0.007% w/v).

In some embodiments, the compositions disclosed herein further comprise an adjuvant. An adjuvant refers to one or more substances that cause stimulation of the immune system. In this context, an adjuvant is used to enhance an immune response to one or more vaccine antigens or antibodies. An adjuvant may be administered to a subject before, in combination with, or after administration of the vaccine. Examples of chemical compounds used as adjuvants include aluminum compounds, oils, block polymers, immune stimulating complexes, vitamins, and minerals (e.g., vitamin E, vitamin A, selenium, and vitamin B12), Quil A (saponins), bacterial and fungal cell wall components (e.g., lipopolysaccharides, lipoproteins, and glycoproteins), hormones, cytokines, and co-stimulatory factors.

Any of the above compositions or formulations disclosed herein may further comprise at least one additional therapeutic agent.

### 4. Methods

The disclosed polypeptide may be useful in a variety of methods. For example, the disclosed polypeptides may be utilized in methods directed to the localization and/or quantitation of a target molecule (e.g., for use in measuring levels of a target molecule (e.g., an antigen, a receptor, a ligand, a substrate etc.) within appropriate physiological samples) in diagnostic or imaging methods. The disclosed polypeptides may be utilized in methods directed to isolating, purifying, or detecting a target molecule in techniques such as affinity chromatography, immunofluorescence, flow cytometry, immunohistochemistry, or immunoprecipitation. The disclosed polypeptides may be particularly useful in prophylactic or therapeutic methods (e.g., to treat or prevent a disease or disorder).

Further disclosed herein are methods of treating disease or disorder, comprising administering to a subject an effective amount of a polypeptide, or a nucleic acid encoding thereof, or composition disclosed herein. The subject may be suffering from, diagnosed as having, or at risk for developing the disease or disorder. In some embodiments, the subject is human.

The effective amount may include a “therapeutically effective amount” or a “prophylactically effective amount” of the polypeptide or nucleic acid encoding thereof. A “therapeutically effective amount” refers to an amount effective, at dosages and for periods of time necessary, to achieve the desired therapeutic result. A therapeutically effective amount may be determined by a person skilled in the art and may vary according to factors such as the disease state, age, sex, and weight of the individual, and the ability of the composition to elicit a desired response in the individual. A therapeutically effective amount is also one in which any toxic or detrimental effects are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The methods are not limited by the type of disease or disorder. Generally, the disclosed polypeptides can be used for treating and preventing diseases or disorders which currently find use in antibody or Fc fusion protein treatment methods, including but not limited to autoimmune diseases, immunological diseases, infectious diseases, inflammatory diseases, neurological diseases, and oncological and neoplastic diseases including cancer.

The disclosed polypeptides, nucleic acids, or compositions may be administered to a subject by a variety of methods. In any of the uses or methods described herein, administration may be by various routes known to those skilled in the art, including without limitation, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis). In some embodiments, the administration is intravenously, intramuscularly, intraarterially, intrathecally, intracapsularly, intraorbitally, intradermally, intraperitoneally, transtracheally, subcutaneously, intracerebroventricularly, orally, intratumorally, intranasally, or as gene therapy.

The disclosed polypeptides, nucleic acids, or compositions may be administered as a single bolus to a subject in need thereof. Alternatively, the dosing regimen may comprise multiple administrations performed at various times after the appearance of tumors.

The disclosed polypeptides, nucleic acids, or compositions can be administered as a single active agent or in combination (a single composition or multiple compositions) with one or more additional active agents including, but not limited to, cytotoxic agents, chemotherapeutic agents, cytokines, growth inhibitory agents, anti-hormonal agents, kinase inhibitors, anti-angiogenic agents, cardioprotectants, or other therapeutic agents.

The amount of the polypeptides, nucleic acids, or compositions of the present disclosure required for use in the disclosed methods will vary not only with the particular polypeptide, nucleic acid, or composition but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages can be determined by comparing their in vitro activity, and in vivo activity in animal models.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

### 5. Kits

In another aspect, the disclosure provides kits comprising a polypeptide as disclosed, a nucleic acid encoding a disclosed polypeptide, or a composition comprising a disclosed polypeptide and instructions for use.

The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed polypeptide, nucleic acid, or composition and/or product and another agent for delivery to a patient.

The kits can also comprise instructions for using the components of the kit. The instructions are relevant materials or methodologies pertaining to the kit. The materials may include any combination of the following: background information, list of components, brief or detailed protocols for using the compositions, troubleshooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. For example, the disclosed kit may further provide a means to administer the disclosed polypeptide, nucleic acid, or composition to the subject, e.g., a pre-filled syringe, a vial and syringe, an injection pen, an auto-injector, a dripper and iv bag, a pump, etc.

The kits provided herein are in suitable packaging. Suitable packaging includes, but is not limited to, vials, bottles, jars, flexible packaging, and the like. Individual member components of the kits may be physically packaged together or separately.

### 6. Examples

### Example 1

### Self-Association of Omalizumab

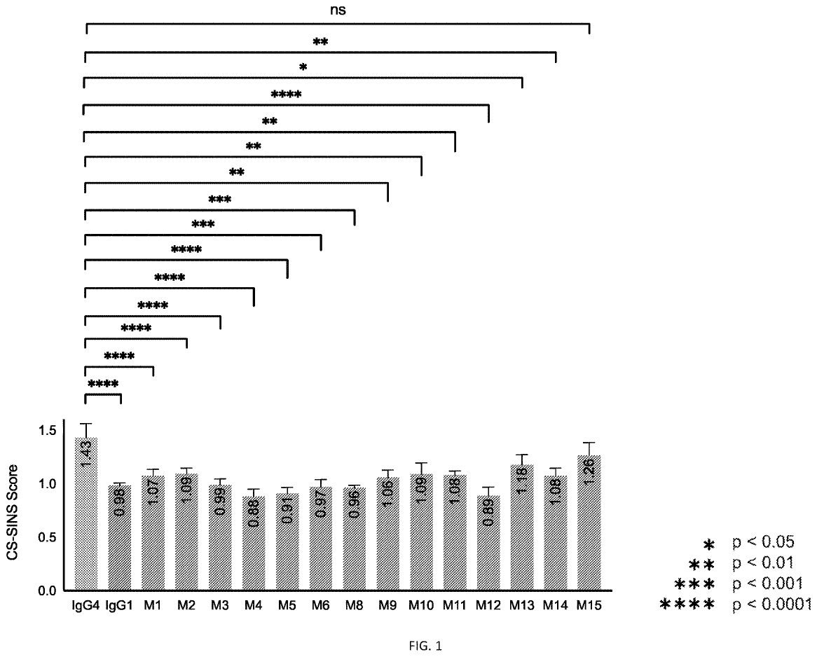

Omalizumab is a recombinant humanized IgG1k monoclonal antibody that specifically binds to free human immunoglobulin E (IgE). The C_{H}3 domain was swapped for that of a wild-type IgG4 and an IgG4 variant having the indicated amino acid substitutions. The self-association was characterized by charge-stabilized self-interaction nanoparticle spectroscopy (CS-SINS), an affinity-capture nanoparticle assay that measures colloidal self-interaction at ultradilute antibody concentration and is predictive of high viscosity and opalescence. For experimental details of CS-SINS see International Patent Publication No. WO2022148777, incorporated herein by reference in its entirety. Results are shown in FIG. 1 and Table 1.

TABLE 1

| | Mutations | | | | | | Melting |
|---|---|---|---|---|---|---|---|
| | | Kabat | Yield | Monomer | CS-SINS | CS-SINS | Temp |
| Mutant# | EU Numbering | Numbering | (mg/L) | (%) | (n = 3) | STDEV | (° C.) |
| Oma G1 WT | — | — | 24.5 | 98 | 0.98 | 0.024 | 80.1 |
| Oma G4 WT | — | — | 18.0 | 82 | 1.43 | 0.13 | 70.1 |
| Mut1 (M1) | Q355R | Q376R | 38.3 | 93 | 1.07 | 0.062 | 68.9 |
| Mut2 (M2) | E419Q | E450Q | 47.0 | 91 | 1.09 | 0.054 | 70.4 |
| Mut3 (M3) | E419R | E450R | 47.5 | 88 | 0.99 | 0.054 | 69.9 |
| Mut4 (M4) | Q355R/L443K | Q376R/L474K | 43.0 | 91 | 0.88 | 0.070 | 72.4 |
| Mut5 (M5) | Q355R/L443R | Q376R/L474R | 36.0 | 91 | 0.91 | 0.054 | 73.8 |
| Mut6 (M6) | E419Q/L443K | E450Q/L474K | 41.3 | 92 | 0.97 | 0.071 | 73.2 |
| Mut7 (M7) | E419Q/L443R | E450Q/L474R | 3.0 | 96 | — | — | |
| Mut8 (M8) | Q355R/E419Q | Q376R/E450Q | 43.1 | 92 | 0.96 | 0.025 | 72.8 |
| Mut9 (M9) | Q355R/Q362K | Q376R/Q385K | 44.0 | 92 | 1.06 | 0.071 | 70.2 |
| Mut10 (M10) | Q355R/E382Q | Q376R/E407Q | 41.8 | 92 | 1.09 | 0.10 | 68.5 |
| Mut11 (M11) | Q355R/N390K | Q376R/N418K | 39.9 | 86 | 1.08 | 0.038 | 69.2 |
| Mut12 (M12) | Q355R/E419R | Q376R/E450R | 37.6 | 95 | 0.89 | 0.081 | 71..2 |
| Mut13 (M13) | E419Q/Q362K | E450Q/Q385K | 31.3 | 88 | 1.18 | 0.093 | 70.6 |
| Mut14 (M14) | E419Q/E382Q | E450Q/E407Q | 36.8 | 85 | 1.08 | 0.071 | 68.2 |
| Mut15 (M15) | E419Q/N390K | E450Q/N418K | 32.9 | 76 | 1.26 | 0.12 | 69.1 |

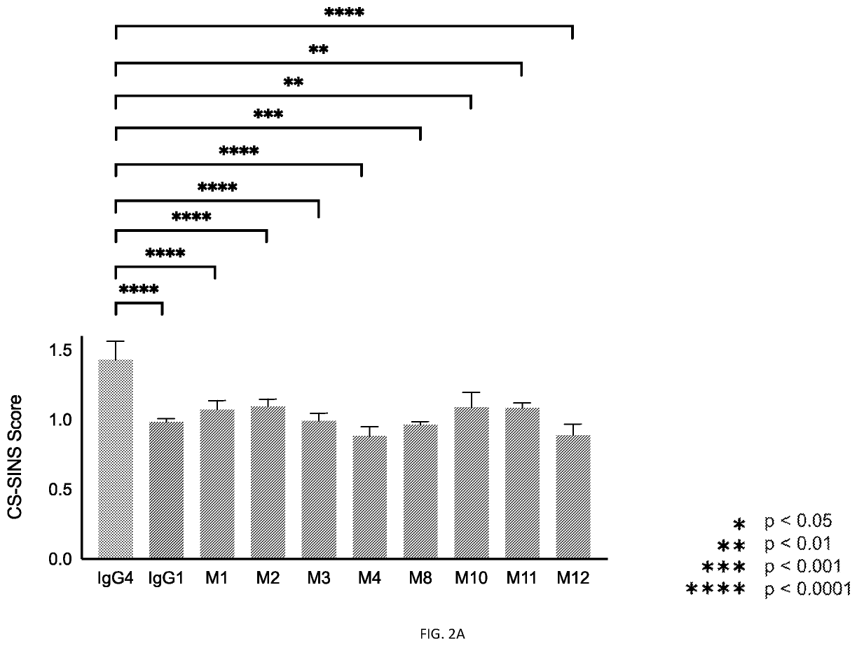

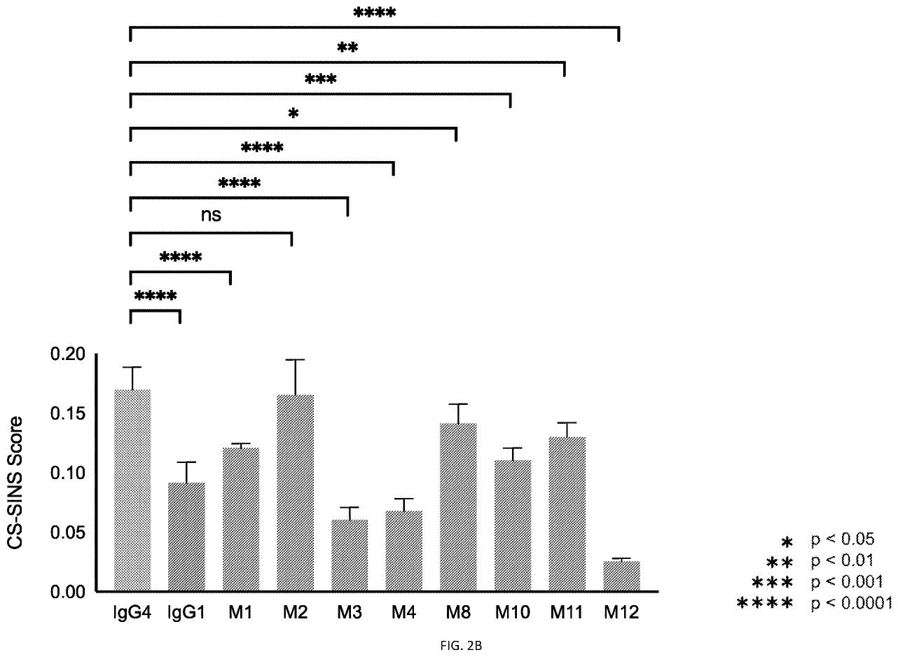

FIGS. 2A and 2B show a comparison of CS-SINS results for select omalizumab IgG4 variants at pH 6 (10 mM histidine) and pH 5 (10 mM acetate), respectively. Thus, many of the mutations that reduce self-association of omalizumab IgG4 at pH 6 result in similar functionality at pH 5.

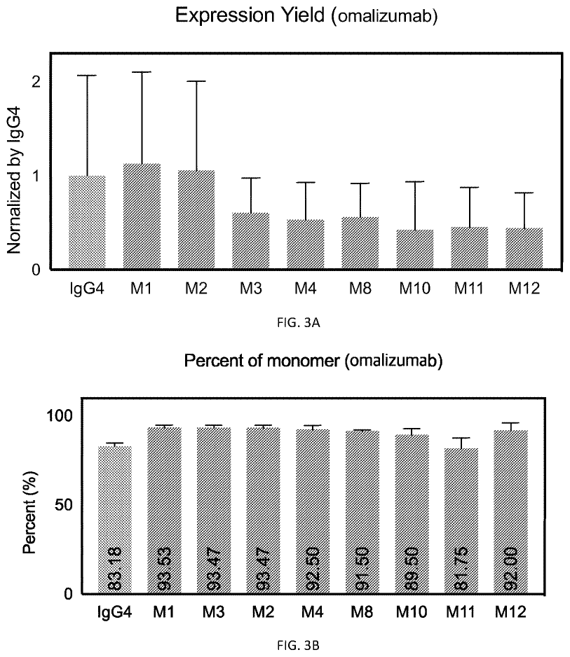

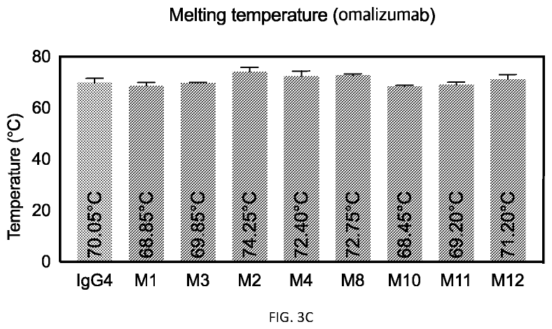

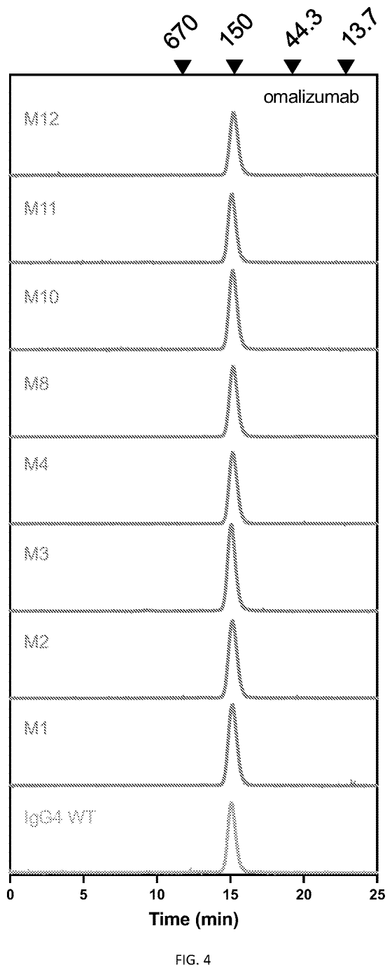

The omalizumab IgG4 variants were analyzed for a number of other biochemical and biophysical characteristics. FIG. 3 shows graphs showing the expression yield, percent monomer and melting temperature of select omalizumab IgG4 variants. FIG. 4 shows the apparent molecular weight and purity analysis using size exclusion chromatography. Overall, the majority of the mutants tested show similar biophysical and biochemical characteristics as compared to wild-type omalizumab.

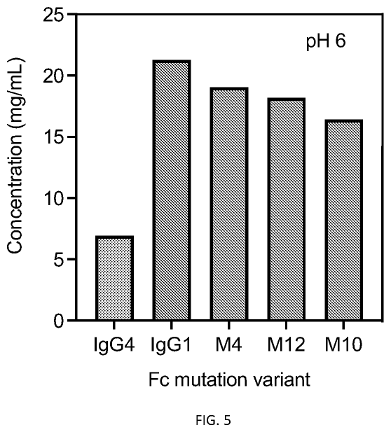

A solubility analysis was performed on wild-type IgG1, wild-type IgG4, and omalizumab IgG4 variant. One milligram of each antibody was centrifuged for about one hour using a 10 kDa molecular weight cut-off centrifugal concentrator. The resulting material was applied to a second 10 kDa molecular weight cut-off centrifugal concentrator and centrifuged for about one hour. The resulting concentration was determined by UV measurements of the sample obtained at three different dilutions into pH 6 buffer. As shown in FIG. 5, Fc mutations increase the solubility of omalizumab IgG4, approaching the level of solubility seen for wild-type IgG1, without affecting the other biochemical and biophysical characteristics analyzed.

### Example 2

### Self-Association of Cabiralizumab and Revdofilimab

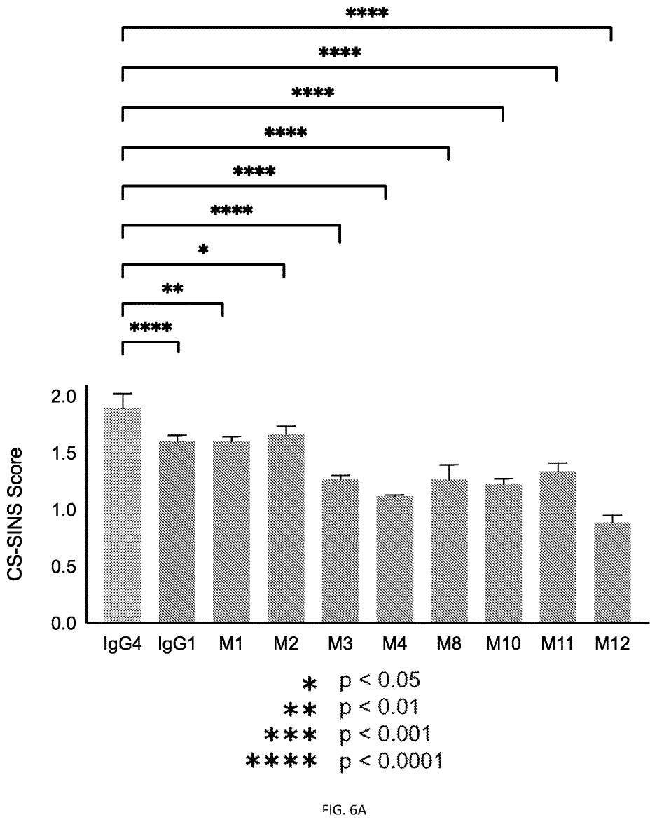

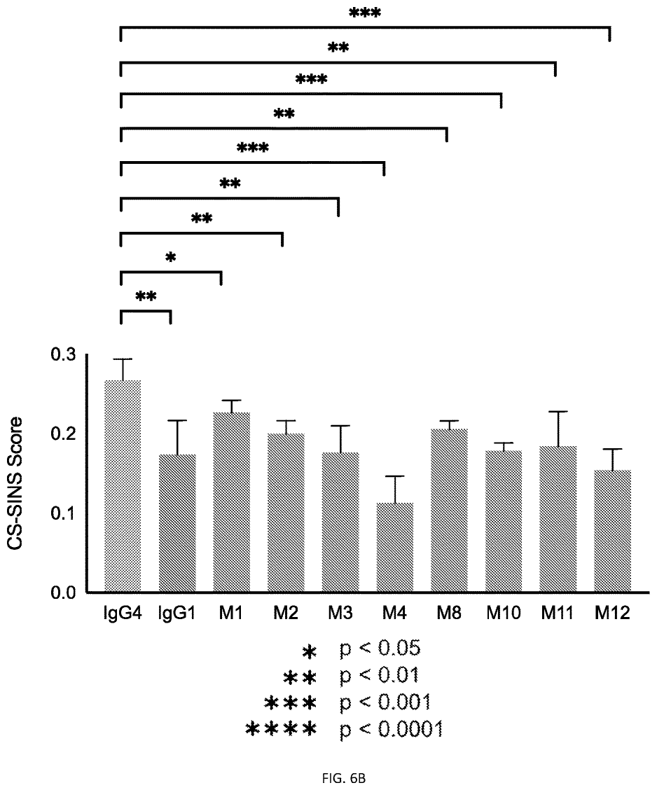

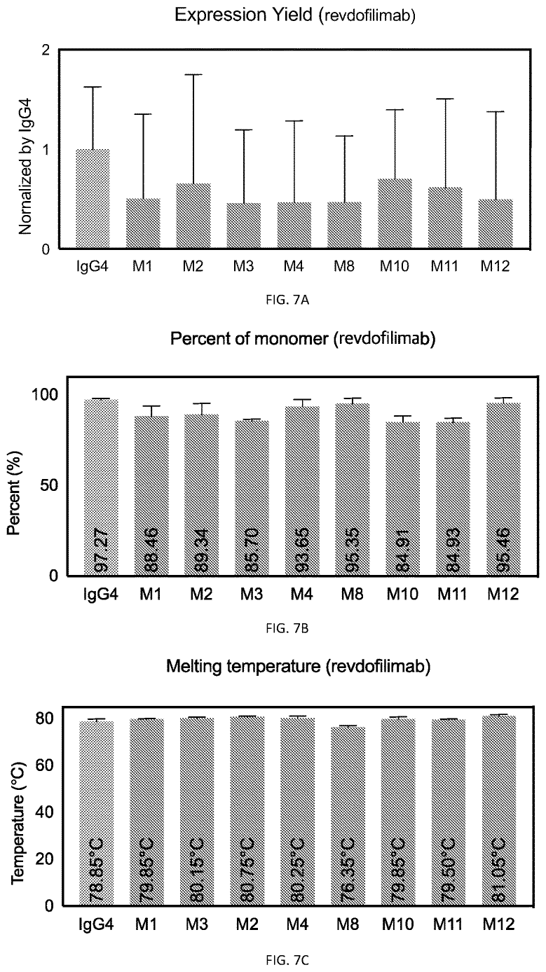

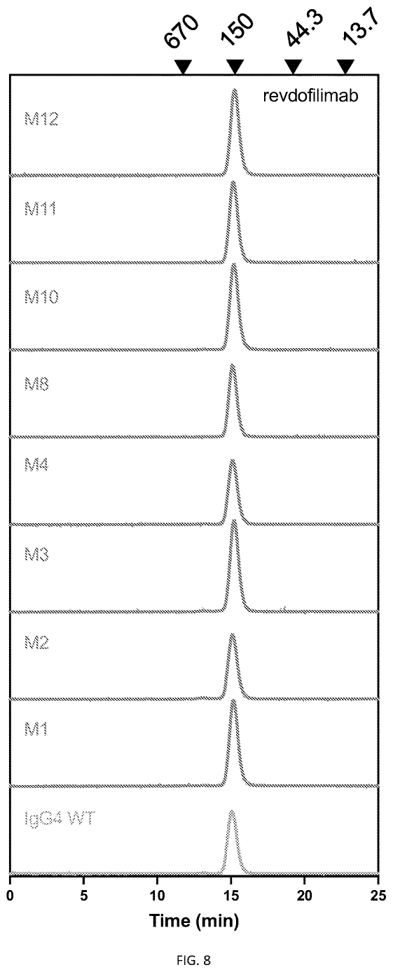

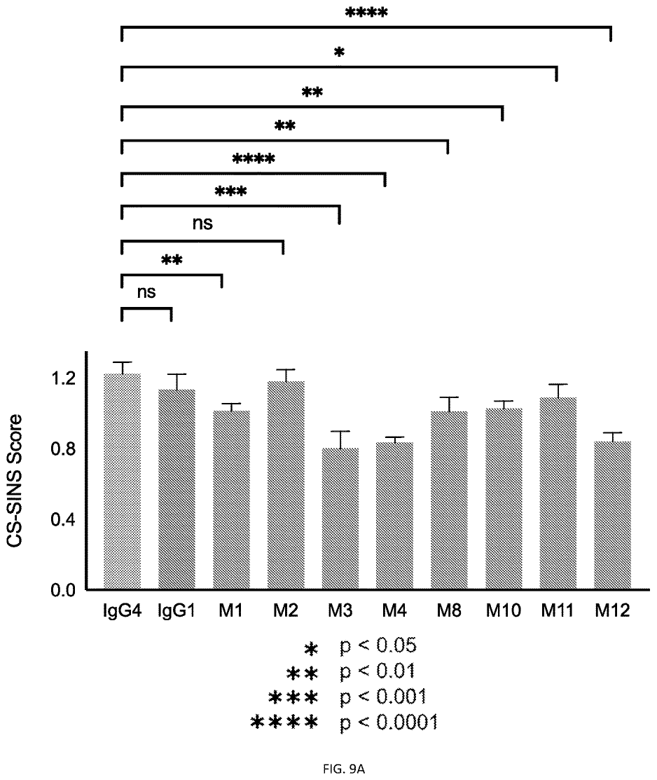

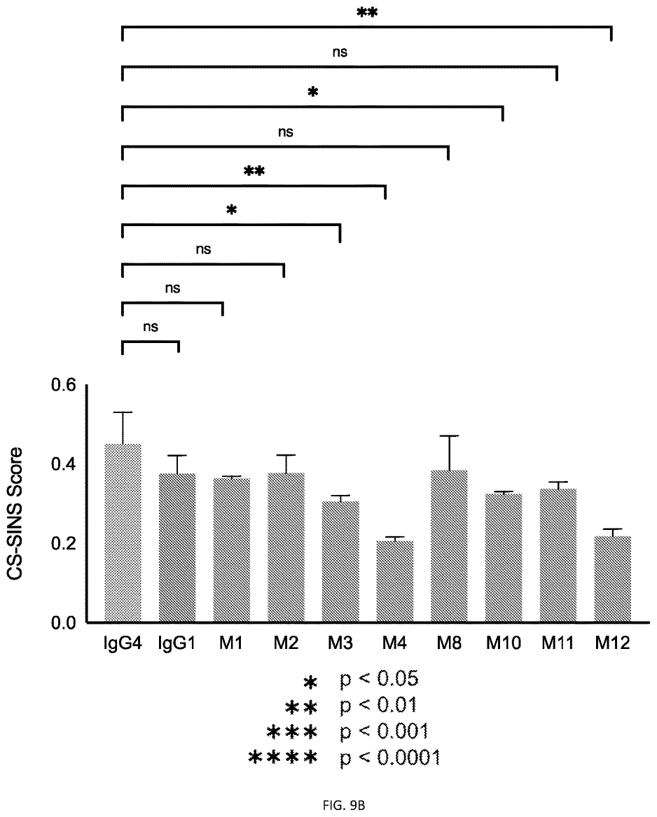

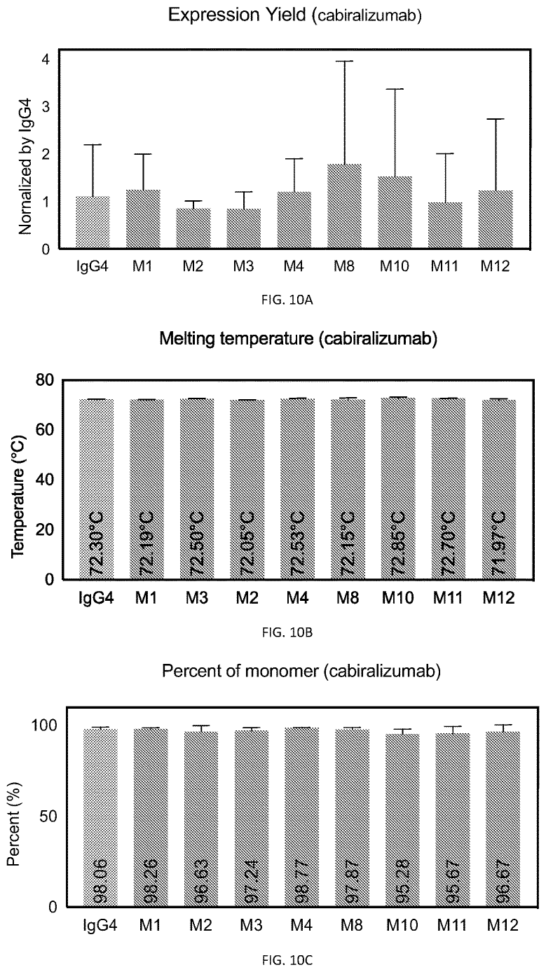

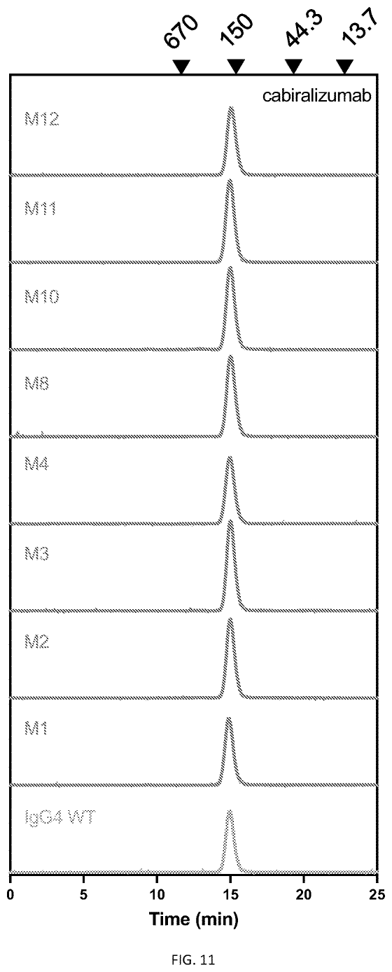

The C_{H}3 domain of other IgG1 isotype antibodies (cabiralizumab and revdofilimab) was swapped with the above variant IgG4 based C_{H}3 domains for characterization of self-association, as shown in FIGS. 6 and 9 for revdofilimab and cabiralizumab, respectively. Similar biochemical and biophysical analysis of the revdofilimab (FIGS. 7-8) and cabiralizumab (FIGS. 10-11) variants affirmed that the described mutations in the C_{H}3 domain did not substantially alter antibody characteristics as compared to wild-type.

### Sequences

For the hinge region sequences, the upper hinge is shown in bold, the core is shown underlined and the lower hinge is in bold underlined.

Omalizumab heavy chain with domains VH, CH1, hinge, CH2, and CH3:
(VH)
(SEQ ID NO: 1)
EVQLVESGGGLVQPGGSLRLSCAVSGYSITSGYSWNWIRQAPGKGLEWVASITYDGSTNYNP
SVKGRITISRDDSKNTFYLQMNSLRAEDTAVYYCARGSHYFGHWHFAVWGQGTLVTVSS
(CH1 EU118-215)
(SEQ ID NO: 2)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRV
(Hinge EU216-230)
(SEQ ID NO: 3)
ESKYGPPCPPCP
(CH2 EU231-340)
(SEQ ID NO: 4)
APEFLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAK
(CH3 EU341-447)
(SEQ ID NO: 5)
GQPREPQVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
Omalizumab light chain with domains VL, CL:
(VL)
(SEQ ID NO: 6)
DIQLTQSPSSLSASVGDRVTITCRASQSVDYDGDSYMNWYQQKPGKAPKLLIYAASYLESGVP
SRFSGSGSGTDFTLTISSLQPEDFATYYCQQSHEDPYTFGQGTKVEIKR
(CL)
(SEQ ID NO: 7)
TVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
Revdofilimab heavy chain with domains VH, CH1, hinge, CH2 and CH3:
(VH)
(SEQ ID NO: 8)
EVQLVESGGGLVQPGGSLRLSCAASGFTFSRYGMSWVRQAPGKGLELVATINSNGGRTYYPD
SVKGRFTISRDNAKNSLYLQMNSLRAEDTAVYYCAREGITTAYAMDYWGQGTTVTVSS
(CH1 EU118-215)
(SEQ ID NO: 9)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRV
(Hinge EU216-230)
(SEQ ID NO: 10)
ESKYGPPCPPCP
(CH2 EU231-340)
(SEQ ID NO: 11)
APEFLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAK
(CH3 EU341-447)
(SEQ ID NO: 12)
GQPREPQVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
Revdofilimab light chain with domains VL, CL:
(VL)
(SEQ ID NO: 13)
DIVMTQSPDSLAVSLGERATINCKASQSVDYDGDSYMHWYQQKPGQPPKLLIYAASILESGVP
DRFSGSGSGTDFTLTISSLQAEDVAVYYCQQSNEDPRTFGGGTKVEIKR
(CL)
(SEQ ID NO: 14)
TVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
Cabiralizumab heavy chain with domains VH, CH1, hinge, CH2 and CH3:
(VH)
(SEQ ID NO: 15)
QVQLVQSGAEVKKPGSSVKVSCKASGYTFTDNYMIWVRQAPGQGLEWMGDINPYNGGTTFN
QKFKGRVTITADKSTSTAYMELSSLRSEDTAVYYCARESPYFSNLYVMDYWGQGTLVTVSS
(CH1 EU118-215)
(SEQ ID NO: 16)
ASTKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRV
(Hinge EU216-230)
(SEQ ID NO: 17)
ESKYGPPCPPCP
(CH2 EU231-340)
(SEQ ID NO: 18)
APEFLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAK
(CH3 EU341-447)
(SEQ ID NO: 19)
GQPREPQVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
Cabiralizumab light chain with domains VL, CL:
(VL)
(SEQ ID NO: 20)
EIVLTQSPATLSLSPGERATLSCKASQSVDYDGDNYMNWYQQKPGQAPRLLIYAASNLESGIP
ARFSGSGSGTDFTLTISSLEPEDFAVYYCHLSNEDLSTFGGGTKVEIK
(CL)
(SEQ ID NO: 21)
TVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC

| | | SEQ ID |
|---|---|---|
| Description | Sequence (substitution bold/underlined) | NO |
| IgG4 C_{H}3 | GQPREPQVYTLPPSREEMTKNQVSLTCLVKGFYPSDIAVEWESNGQPE | 22 |
| for M1 | NNYKTTPPVLDSDGSFFLYSRLTVDKSRWQEGNVFSCSVMHEALHNHY | |
| | TQKSLSLSLGK | |
| IgG4 C_{H}3 | GQPREPQVYTLPPSQEEMTKNQVSLTCLVKGFYPSDIAVEWESNGQPE | 23 |
| for M2 | NNYKTTPPVLDSDGSFFLYSRLTVDKSRWQQGNVFSCSVMHEALHNH | |
| | YTQKSLSLSLGK | |
| IgG4 C_{H}3 | GQPREPQVYTLPPSQEEMTKNQVSLTCLVKGFYPSDIAVEWESNGQPE | 24 |
| for M3 | NNYKTTPPVLDSDGSFFLYSRLTVDKSRWQRGNVFSCSVMHEALHNH | |
| | YTQKSLSLSLGK | |
| IgG4 C_{H}3 | GQPREPQVYTLPPSREEMTKNQVSLTCLVKGFYPSDIAVEWESNGQPE | 25 |
| for M4 | NNYKTTPPVLDSDGSFFLYSRLTVDKSRWQEGNVFSCSVMHEALHNHY | |
| | TQKSLSKSLGK | |
| IgG4 C_{H}3 | GQPREPQVYTLPPSREEMTKNQVSLTCLVKGFYPSDIAVEWESNGQPE | 26 |
| for M8 | NNYKTTPPVLDSDGSFFLYSRLTVDKSRWQQGNVFSCSVMHEALHNH | |
| | YTQKSLSLSLGK | |
| IgG4 C_{H}3 | GQPREPQVYTLPPSREEMTKNQVSLTCLVKGFYPSDIAVEWQSNGQPE | 27 |
| for M10 | NNYKTTPPVLDSDGSFFLYSRLTVDKSRWQEGNVFSCSVMHEALHNHY | |
| | TQKSLSLSLGK | |
| IgG4 C_{H}3 | GQPREPQVYTLPPSREEMTKNQVSLTCLVKGFYPSDIAVEWESNGQPE | 28 |
| for M11 | NKYKTTPPVLDSDGSFFLYSRLTVDKSRWQEGNVFSCSVMHEALHNH | |
| | YTQKSLSLSLGK | |
| IgG4 C_{H}3 | GQPREPQVYTLPPSREEMTKNQVSLTCLVKGFYPSDIAVEWESNGQPE | 29 |
| for M12 | NNYKTTPPVLDSDGSFFLYSRLTVDKSRWQRGNVFSCSVMHEALHNH | |
| | YTQKSLSLSLGK | |

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

1. A polypeptide comprising a variant IgG Fc region comprising one or more amino acid substitutions at positions selected from 355, 362, 382, 390, 419, and 443, in reference to a wild-type or parent IgG Fc region sequence and numbered according to the EU numbering system.

2. (canceled)

3. The polypeptide of claim 1, wherein the variant IgG Fc region comprises a substitution at position 355 and/or 419.

4. The polypeptide of claim 3, wherein the variant IgG Fc region comprises an amino acid substitution at position 355 and an amino acid substitution at one of positions 362, 382, 390, 419, or 443, or wherein the variant IgG Fc region comprises an amino acid substitution at position 419 and an amino acid substitution at one of positions 355, 362, 382, 390, or 443.

5. The polypeptide of claim 1, wherein:
the amino acid at position 355 of the variant IgG Fc region is substituted with a positively charged amino acid;
the amino acid at position 362 of the variant IgG Fc region is substituted with a positively charged amino acid;
the amino acid at position 382 of the variant IgG Fc region is substituted with a glutamine;
the amino acid at position 390 of the variant IgG Fc region is substituted with a positively charged amino acid;
the amino acid at position 419 of the variant IgG Fc region is substituted with a non-charged or positively charged amino acid; and/or
the amino acid at position 443 of the variant IgG Fc region is substituted with a positively charged amino acid.

6-9. (canceled)

10. The polypeptide of claim 5, wherein:
the amino acid at position 355 of the variant IgG Fc region is substituted with an arginine;
the amino acid at position 419 of the variant IgG Fc region is substituted with an arginine or glutamine;
the amino acid at position 362 of the variant IgG Fc region is substituted with a lysine;
the amino acid at position 390 of the variant IgG Fc region is substituted with a lysine; and/or
the amino acid at position 443 of the variant IgG Fc region is substituted with an arginine or lysine.

11-17. (canceled)

18. The polypeptide of claim 1, wherein the variant IgG Fc region comprises amino acid substitutions selected from:
a) 355R;
b) 419Q;
c) 419R;
d) 355R and 443K or 443R;
e) 355R and 419Q or 419R;
f) 355R and 362K;
g) 355R and 382Q;
h) 355R and 390K;
i) 419Q and 443K or 443R;
j) 419Q and 362K;
k) 419Q and 382Q; and
l) 419Q and 390K.

19. The polypeptide of claim 1, wherein the variant IgG Fc region is derived from a human IgG Fc region.

20. The polypeptide of claim 1, wherein the variant IgG Fc region is an IgG4 Fc region.

21. The polypeptide of claim 1, wherein the polypeptide further comprises a target molecule binding domain.

22. The polypeptide of claim 1, wherein the polypeptide is an antibody or an Fc fusion protein.

23. (canceled)

24. The polypeptide of claim 1, wherein the polypeptide is conjugated to a drug, a toxin, a detectable label, a tag, or a combination thereof.

25. The polypeptide of claim 1, wherein the polypeptide has reduced self-association characteristics.

26. A composition comprising the polypeptide of claim 1 and a pharmaceutically acceptable carrier.

27. The composition of claim 26, wherein the composition is a liquid composition.

28. The composition of claim 27, wherein the composition comprises at least 10 mg/mL of the polypeptide.

29. (canceled)

30. The composition of claim 26, further comprising an amino acid, a tonicity agent, an antioxidant, a stabilizer, an adjuvant, or a combination thereof.

31. The composition of claim 30, wherein the amino acid comprises histidine.

32. The composition of claim 26, wherein the composition has a pH of about 5 to about 7.

33-35. (canceled)

36. A method of treating or preventing a disease or disorder in a subject, comprising administering to the subject an effective amount of the polypeptide of claim 1, a composition comprising the polypeptide, or a nucleic acid encoding the polypeptide.

37. A method of decreasing the self-association of a polypeptide comprising an IgG Fc region comprising introducing one or more amino acid substitutions in the IgG Fc region at positions selected from 355, 362, 382, 390, 419, 443, in reference to a wild-type IgG Fc and numbered according to the EU numbering system or substituting the IgG Fc region with a variant IgG Fc region comprising one or more amino acid substitutions at positions selected from 355, 362, 382, 390, 419, 443, in reference to a wild-type IgG Fc and numbered according to the EU numbering system.

38-45. (canceled)
